(19) United States
(12) Patent Application Publication
(10) Pub. No.: US 2025/0120638 A1
(43) Pub. Date: Apr. 17, 2025

(54) MOISTURE-ABSORBING DRY ELECTRODE AND SMART CLOTHING

(71) Applicant: MAKALOT INDUSTRIAL CO., LTD., Taipei City (TW)

(72) Inventors: Wei-Yi TSAI, Taipei City (TW); Li-Xiang LEE, Taipei City (TW); Chih-Wei CHIU, Taipei City (TW); Shih-Wei HSIEH, Taipei City (TW); Jia-Wun LI, Taipei City (TW)

(21) Appl. No.: 18/616,846

(22) Filed: Mar. 26, 2024

(30) Foreign Application Priority Data
Oct. 13, 2023 (TW) 112139248

(51) Int. Cl. A61B 5/27

(52) U.S. Cl. CPC A61B 5/27

(57) ABSTRACT

A moisture-absorbing dry electrode and smart clothing. The moisture-absorbing dry electrode includes a substrate and a moisture-absorbing electrode. The substrate has a surface. The moisture-absorbing electrode includes at least one moisture-absorbing conductive film, and the moisture-absorbing conductive film is used to absorb moisture and disposed on the surface of the substrate. The material of the moisture-absorbing conductive film includes a uniform mixture of carbon paste, a moisture-absorbing material and a cross-linking agent, and the moisture-absorbing material includes a nanomaterial or an inorganic adsorbent material, or a combination thereof.

### CROSS REFERENCE TO RELATED APPLICATIONS

This Non-provisional application claims priority under 35 U.S.C. § 119(a) on Patent Application No(s). 112139248 filed in Taiwan, Republic of China on Oct. 13, 2023, the entire contents of which are hereby incorporated by reference.

### BACKGROUND

### Technology Field

The present disclosure relates to a dry electrode for absorbing moisture and, in particular, to a moisture-absorbing dry electrode applied to measure physiological signals and a smart clothing configured with the moisture-absorbing dry electrode.

### Description of Related Art

The smart clothing is an innovative product integrating technologies of “electronic components” and “textiles” to allow textiles to meet the expected interactive functions of users, such as the acquisition of physiological signals from the user's body, reminder of the external environment, and provision of external information, etc. In addition, with the development of Internet of Things (IoT), wearable devices have become one of the important applications and development fields. The wearable device is an accessory worn on the human body. In order to achieve the comfortable wearing experience and eliminate foreign object sensations, the material requirements for the electrodes (sensors) in contact with the human skin have been focused on soft, flexible, washable, and biocompatible, and the likes. Moreover, the human skin conductance signals, such as brainwave signals (e.g. EEG), electrocardiograms (ECG), electromyography signals (EMG), etc., are mostly weak electricity signals. Therefore, the electrode patches used to measure skin conductance signals all need to have the low impedance characteristics.

The conventional electrode patches in contact with skin can generally be divided into wet electrodes and dry electrodes. In order to maintain the characteristics of low impedance, soft and conformable, most wet electrodes utilize the conductive adhesive to fit the skin. For example, the wet electrodes include silver chloride patch electrodes or EEG paste electrodes. Although the effect of these wet electrodes is good, the paste or gel will remained on the skin. Therefore, the users must clean and wipe the residual paste or gel later, which leads to inconvenience of users. Moreover, it is also quite disadvantageous for the promotion of the device with the wet electrodes. In addition, the conventional wet electrodes require additional electrolytes or gel-like substances to enhance the contact between the electrodes and the skin, and the long-term wearing may cause skin irritation or allergic reaction. On the contrary, the dry electrodes do not require the use of gel-like substances and are more convenient and stable in use. However, the signal reception of the dry electrode is worse than that of wet electrode, and the signal stability of the dry electrode is poor and is easily interfered by noises.

Therefore, it is desired to provide an electrode patch and a smart clothing with the electrode patch that have the convenience and stability as the conventional dry electrodes and the good signal reception and skin comfortable as the conventional wet electrodes.

### SUMMARY

In view of the foregoing, an objective of this disclosure is to provide a moisture-absorbing dry electrode and a smart clothing with the moisture-absorbing dry electrode that can effectively improve the contact stability and signal detection accuracy between the electrode and the skin, thereby achieving more accurate bioelectric signal detection.

To achieve the above, a moisture-absorbing dry electrode of this disclosure includes a substrate and a moisture-absorbing electrode. The substrate has a surface. The moisture-absorbing electrode includes at least one moisture-absorbing conductive film configured to absorb moisture, and the moisture-absorbing conductive film is disposed on the surface of the substrate. The material of the moisture-absorbing conductive film includes a uniform mixture of carbon paste, a moisture-absorbing material and a cross-linking agent, and the moisture-absorbing material includes a nanomaterial or an inorganic adsorbent material, or a combination thereof.

To achieve the above, a smart clothing of this disclosure, which is applied to measure a physiological signal, includes a clothing body and a moisture-absorbing dry electrode. The clothing body is configured to be worn on a human body. The moisture-absorbing dry electrode is arranged at an inner side of the clothing body and configured to contact the human body for measuring the physiological signal. The moisture-absorbing dry electrode includes a substrate and a moisture-absorbing electrode. The substrate is attached to the clothing body and has a surface away from the clothing body. The moisture-absorbing electrode includes at least one moisture-absorbing conductive film configured to absorb moisture and is disposed on the surface of the substrate. The material of the moisture-absorbing conductive film includes a uniform mixture of carbon paste, a moisture-absorbing material and a cross-linking agent, and the moisture-absorbing material includes a nanomaterial or an inorganic adsorbent material, or a combination thereof.

In one embodiment, the material of the substrate includes a polymer material.

In one embodiment, the carbon paste is a mixture of a solvent and one or any combinations of graphene, carbon nanotubes and carbon black.

In one embodiment, the content of the moisture-absorbing material is between 0.1 and 50 weight percentage of the carbon paste.

In one embodiment, the nanomaterial includes attapulgite, silicon dioxide, zeolite, porous carbon, activated carbon, covalent organic framework material, metal organic framework material or porous organic polymer, or any combinations thereof.

In one embodiment, the inorganic adsorbent material includes metal salt or activated alumina, or a combination thereof.

In one embodiment, the thickness of the moisture-absorbing conductive film is between 1 μm and 2000 μm.

In one embodiment, the moisture-absorbing electrode includes multiple moisture-absorbing conductive films, the moisture-absorbing conductive films are stacked and arranged on the surface of the substrate, and the total thickness of the moisture-absorbing conductive films is between 1 μm and 2000 μm.

In one embodiment, the moisture-absorbing material includes lithium chloride, and the moisture-absorbing ability of the moisture-absorbing electrode has a positive correlation with the content of the lithium chloride in the moisture-absorbing material.

As mentioned above, in the moisture-absorbing dry electrode and smart clothing of the present disclosure invention, the conductive film is added with the moisture-absorbing material for absorbing moisture, so that the contact area between the electrode and human skin can be increased, thereby improving signal reception. Compared with the conventional electrode patches, the moisture-absorbing dry electrode and smart clothing of this disclosure have the convenience and stability as the conventional dry electrodes and the good signal reception and skin comfortable as the conventional wet electrodes, so that they can be conveniently used and do not require additional electrolysis or gel-like substance. Therefore, the long-term wearing of the smart clothing will not cause skin irritation or allergic reactions. In addition, the moisture-absorbing dry electrode of the present disclosure has good stability, so that it can achieve better resistance to movement or other interferences, and can maintain signal quality more stably. Moreover, in high-activity scenes such as sports and fitness, the moisture-absorbing material can absorb sweat and other secretions to maintain good contact between the electrode patch and the skin, thereby ensuring signal quality and stability and meeting the needs of users.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 and FIG. 2 are schematic diagrams showing moisture-absorbing dry electrodes according to different embodiments of this disclosure;

FIGS. 3A to 3E are schematic diagrams showing the weight changes of carbon paste and different moisture-absorbing materials (with different proportions) in room temperature during the moisture absorption according to an embodiment of the present disclosure;

FIGS. 4A to 4C are schematic diagrams showing the results of the moisture absorption cycle test of a moisture-absorbing conductive film made of carbon paste and moisture-absorbing materials in different proportions;

FIGS. 5A and 5B are schematic diagrams showing the results of the moisture-absorbing effects of carbon paste and different moisture-absorbing materials (with different proportions) in different humidity environments;

FIG. 6 is a schematic diagram showing the electrocardiogram (ECG) signals measured by the electrode patches made of different materials and proportions;

FIG. 7 is a schematic diagram of the signal-to-noise ratio plotted from the measurement results of FIG. 6; and

FIG. 8 is a schematic diagram showing a part of the smart clothing according to an embodiment of the present disclosure.

### DETAILED DESCRIPTION OF THE DISCLOSURE

The present disclosure will be apparent from the following detailed description, which proceeds with reference to the accompanying drawings, wherein the same references relate to the same elements.

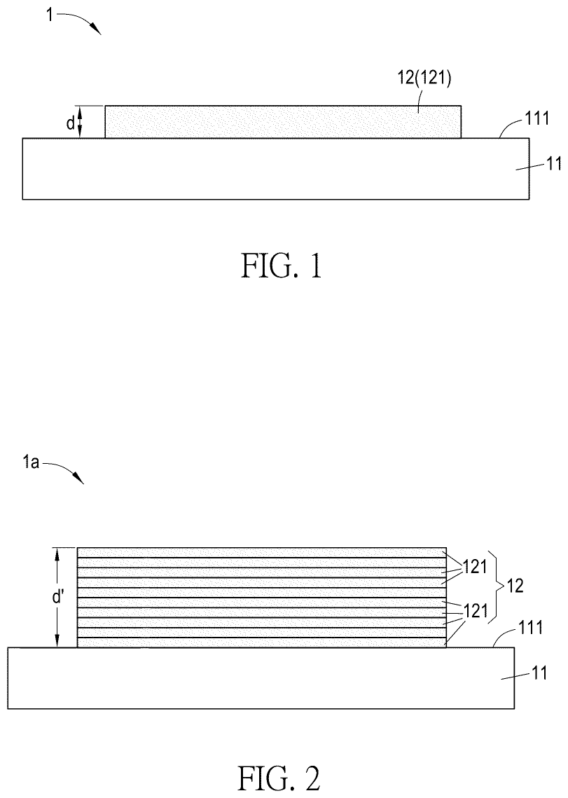

FIG. 1 and FIG. 2 are schematic diagrams showing moisture-absorbing dry electrodes according to different embodiments of this disclosure.

With reference to FIG. 1, a moisture-absorbing dry electrode 1 of this embodiment includes a substrate 11 and a moisture-absorbing electrode 12.

The substrate 11 has a surface 111. In this embodiment, the substrate 11 can be an insulating flexible substrate, which can be made of polymer materials, such as thermoplastic or thermosetting polymer materials. In particular, the polymer material of the substrate 11 can be, for example but not limited to, thermoplastic or thermosetting polyurethane (TPU), sulfonated ethylene-vinyl acetate (SEVS), polyvinylidene fluoride (PVDF) or polyvinyl alcohol (PVA), or any combination thereof. The substrate 11 of this embodiment is a TPU substrate as an example. Generally speaking, the thickness of the substrate 11 is not limited, and is, for example, greater than the thickness of the moisture-absorbing electrode 12.

The moisture-absorbing electrode 12 includes at least one moisture-absorbing conductive film 121, which is configured to absorb moisture, and the moisture-absorbing electrode 12 (the moisture-absorbing conductive film 121) is disposed on the surface 111 of the substrate 11. In this embodiment, the moisture-absorbing electrode 12 includes one layer of the moisture-absorbing conductive film 121. Namely, the moisture-absorbing electrode 12 (the moisture-absorbing conductive film 121) is used to detect skin conductance signals of the human body, such as brainwave signals (e.g. EEG), electrocardiograms (ECG), electromyography signals (EMG), or other weak electric signals. In addition, the moisture-absorbing electrode 12 also has a moisture-absorbing effect and can absorb moisture or water vapor in the environment. This configuration can increase the contact area between the electrode and human skin and reduce contact resistance, thereby improving signal reception (improving signal-to-noise ratio). In this embodiment, the material of the moisture-absorbing conductive film 121 may include a uniform mixture of carbon paste, a moisture-absorbing material and a cross-linking agent.

The carbon paste is a mixture of solvent and carbon material. For example, the carbon paste can be a mixture of ethylene glycol solvent and graphene, carbon nanotubes or carbon black, or a mixture of ethylene glycol solvent and any combination of graphene, carbon nanotubes and carbon black (i.e., a mixture of ethylene glycol solvent and at least one of graphene, carbon nanotubes and carbon black). The purpose of adding the carbon paste is to provide electronic conductivity, and in particular, to detect physiological signals and conduct the physiological signals. In addition, the moisture-absorbing material may include nanomaterial or inorganic adsorbent material, or a combination thereof. In some embodiments, the nanomaterial can be attapulgite, silicon dioxide (e.g. mSiO₂·nH₂O), zeolite, porous carbon, activated carbon, covalent organic framework materials (COFs), metal organic framework materials (MOFs) or porous organic polymers (POPs), or any combinations thereof. In one embodiment, the porous organic polymers (POPs) include crystalline porous organic polymers or amorphous porous organic polymers. In one embodiment, the inorganic adsorbent material may be a metal salt or activated alumina, or a combination thereof. In one embodiment, the metal salt may be, for example, lithium bromide (LiBr), lithium chloride (LiCl), potassium bromide (KBr), calcium chloride (CaCl₂) or magnesium chloride (MgCl₂).

The purpose of adding the moisture-absorbing material is to absorb moisture from the environment and increase the contact area between the electrode and the skin, thereby improving signal reception. When the moisture-absorbing dry electrode is applied (e.g. worn on the user), the moisture-absorbing material can also absorb sweat and body secretions to maintain good contact between the electrode patch and the skin, thereby ensuring signal quality and stability. In particular, lithium chloride has good chemical stability, thermal stability, and moisture-absorbing property, so that it can absorb moisture from the environment. Attapulgite, also known as palygorskite or palygorskite, is a water-containing magnesium-rich aluminum silicate clay mineral with a chain layered structure that can also absorb water from the environment. Silicon dioxide is a granular porous silica hydrate that also has good water absorption ability. In addition, a cross-linking agent is a substance that can play a bridging role between linear molecules, thereby bonding and cross-linking multiple linear molecules to each other to form a network structure. Common cross-linking agents include, for example, organic peroxides.

In some embodiments, the content of the moisture-absorbing material is between 0.1 and 50 weight percentage (wt %) of the carbon paste. In some embodiments, the content of the moisture-absorbing material is between 0.1 wt % and 10 wt % of the carbon paste, such as 0.1 wt %, 0.3 wt %, 0.5 wt %, 0.8 wt %, 0.9 wt %, 1.0 wt %, 3 wt %, 5 wt %, 10 wt %, etc. In some embodiments, the thickness d of the moisture-absorbing conductive film 121 may range from 1 to 2000 microns (μm), such as 10 μm, 50 μm, 150 μm, 200 μm, 500 μm, 800 μm, 1000 μm, 1250 μm or 1500 μm.

The structure and feature of the moisture-absorbing dry electrode 1a of FIG. 2 are mostly the same as those of the moisture-absorbing dry electrode 1 of FIG. 1. Unlike the moisture-absorbing dry electrode 1, the moisture-absorbing electrode 12 of the moisture-absorbing dry electrode 1a of this embodiment includes a plurality (e.g. ten) of the moisture-absorbing conductive films 121, which are stacked (laminated) on the surface 111 of the substrate 11. In this embodiment, the total thickness d′ of the moisture-absorbing conductive films 121 ranges from 1 μm to 2000 μm. In some embodiments, the moisture-absorbing electrode 12 may include 2, 3 or 5 stacked (laminated) moisture-absorbing conductive films 121. In some embodiments, the moisture-absorbing electrode 12 may include more than 10 stacked (laminated) moisture-absorbing conductive films 121.

In some embodiments, the production process of the moisture-absorbing dry electrode may, for example, include the following steps. 6 g of carbon paste containing carbon nanotubes is added with a certain proportion of a moisture-absorbing materials (e.g. 0.06 g (i.e. 1 wt % of the carbon paste) of the moisture-absorbing material, such as lithium chloride, attapulgite or silicon dioxide particles). The mixture is dissolved or dispersed in 6 g ethylene glycol solution, and then added with a cross-linking agent (e.g. 1 wt % of the carbon paste). The solution is stirred and well mixed with a planetary degassing mixer to obtain a gel. The gel is then coated on the TPU film (substrate 11) by using a blade, which is then placed in an oven and dried at 50° C. to obtain the moisture-absorbing conductive film 121. In one embodiment, the thickness of the gel coated on the TPU film is about 1000 μm, and the thickness of the obtained moisture-absorbing conductive film 121 after drying is between 60 and 100 m. In another embodiment, in the production process of the moisture-absorbing dry electrode 1a of FIG. 2, after obtaining one layer of the moisture-absorbing conductive film 121, the coating and drying steps can be repeated to form a plurality of stacked (laminated) moisture-absorbing conductive films 121 on the surface 111 of the substrate 11. It can be understood that the above-mentioned production process of the moisture-absorbing dry electrode (or moisture-absorbing conductive film) is just an example. In different embodiments, different processes can be used to obtain the moisture-absorbing dry electrode (or moisture-absorbing conductive film) with the same structure, and this disclosure is not limited.

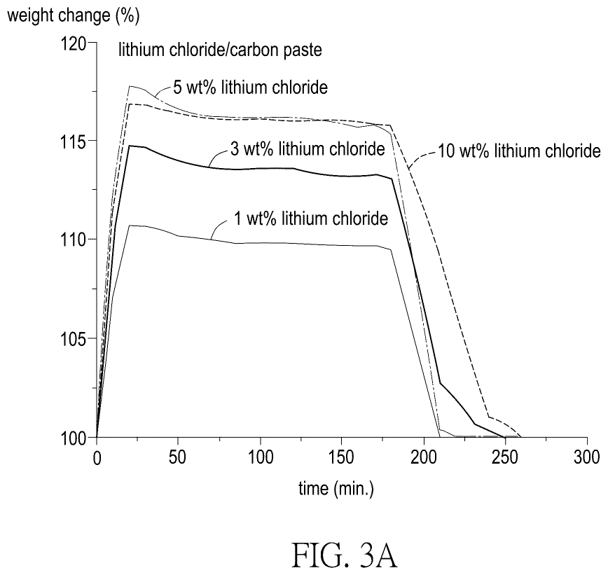

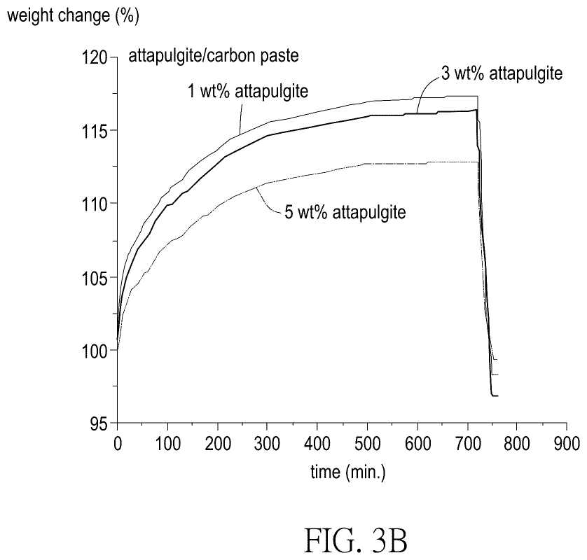

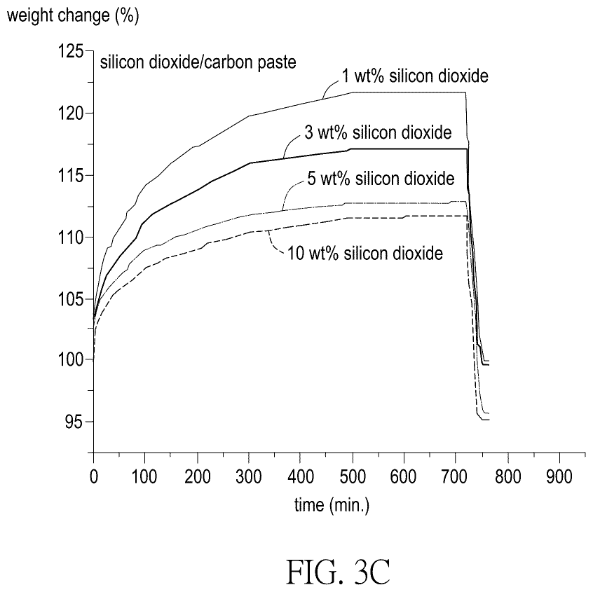

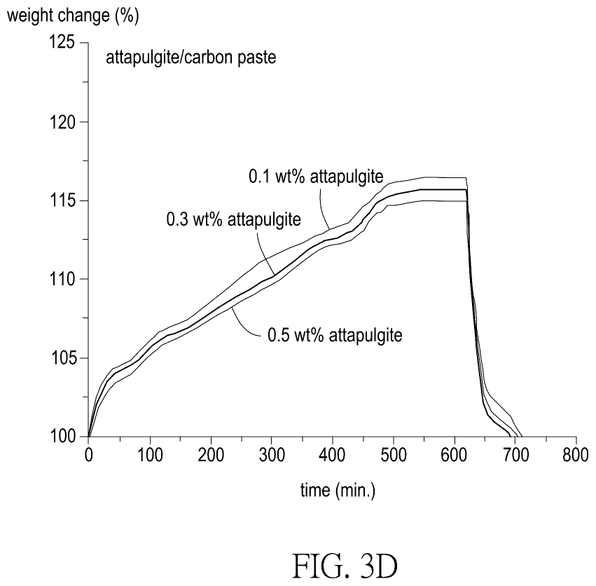

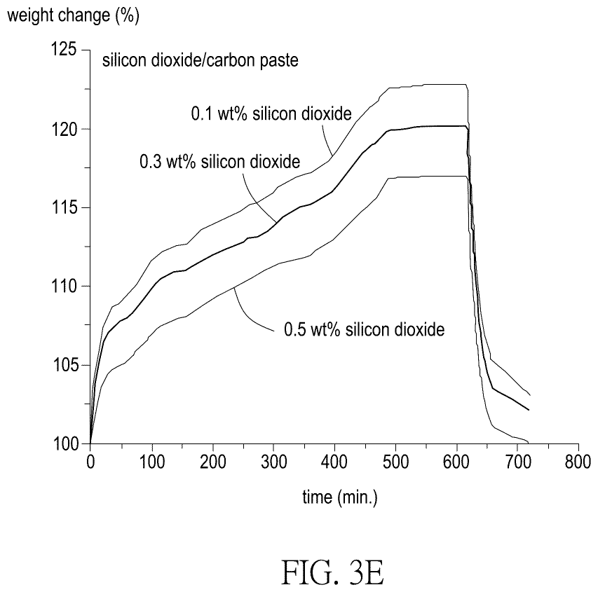

FIGS. 3A to 3E are schematic diagrams showing the weight changes of carbon paste and different moisture-absorbing materials (with different proportions) in room temperature (25° C., 60% RH) during the moisture absorption according to an embodiment of the present disclosure. FIG. 3A shows the weight changes of carbon paste and lithium chloride (with different proportions), FIGS. 3B and 3D show the weight changes of carbon paste and attapulgite (with different proportions), and FIGS. 3C and 3E show the weight changes of carbon paste and silicon dioxide (with different proportions). To be noted, the production process of the moisture-absorbing conductive film used in the example of FIG. 3B is different from that of FIG. 3D, and the production process of the moisture-absorbing conductive film used in the example of FIG. 3C is different from that of FIG. 3E.

As shown in FIG. 3A, when the content of lithium chloride is below 5 wt % (including 5 wt %), the increase of the concentration of lithium chloride will increase the moisture-absorbing effect. That is, the moisture-absorbing effect is in positive correlation with the content of lithium chloride. In addition, when the content of lithium chloride is above 5 wt % (e.g. 10 wt %), the increase of the concentration of lithium chloride will cause little change in the moisture-absorbing effect. As shown in FIGS. 3B to 3E, when the contents of attapulgite and silicon dioxide increase, the presented moisture-absorbing effects (the weight changes) become worse. In addition, comparing FIGS. 3A, 3B and 3C, it can be seen that, under the same content and the same moisture-absorbing effect, the example with lithium chloride refers to a shorter time than the example with attapulgite or silicon dioxide. That is, the example with lithium chloride can absorb a certain amount of moisture in a shorter period of time, and the moisture-absorbing effect thereof is obviously better.

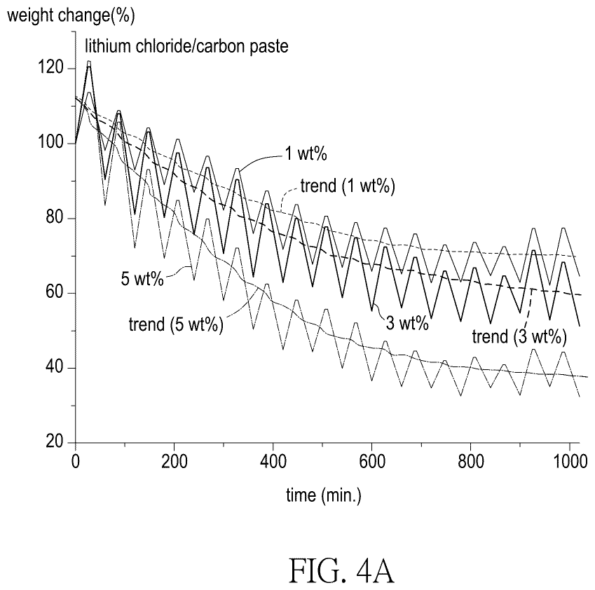

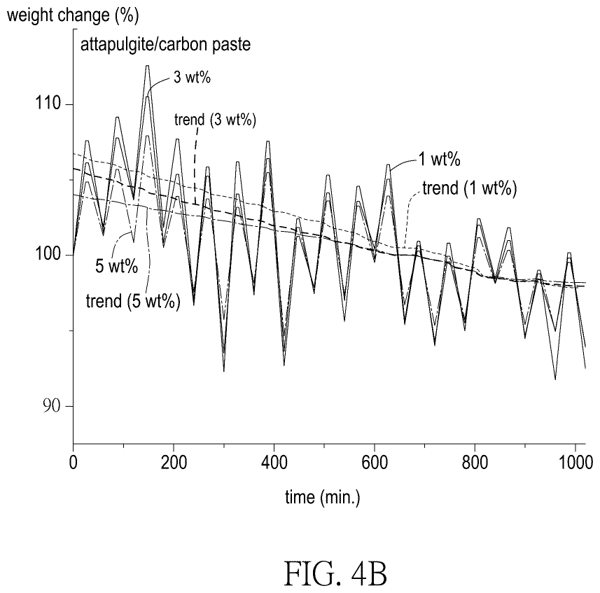

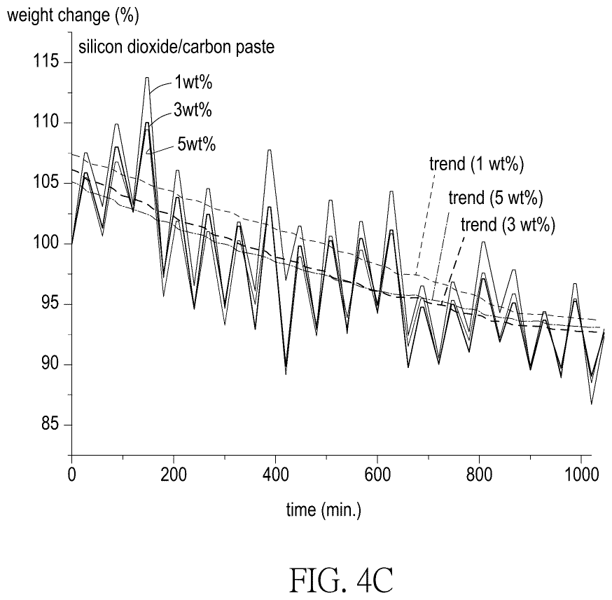

FIGS. 4A to 4C are schematic diagrams showing the results of the moisture absorption cycle test of a moisture-absorbing conductive film made of carbon paste and moisture-absorbing materials in different proportions. FIG. 4A shows the results of the moisture absorption cycle test of a moisture-absorbing conductive film made of carbon paste and lithium chloride in different proportions, FIG. 4B shows the results of the moisture absorption cycle test of a moisture-absorbing conductive film made of carbon paste and attapulgite in different proportions, and FIG. 4C shows the results of the moisture absorption cycle test of a moisture-absorbing conductive film made of carbon paste and silicon dioxide in different proportions. To be noted, the dotted lines shown in FIGS. 4A, 4B and 4C are respectively the weight change trend curves of carbon paste and different content proportions of lithium chloride, attapulgite and silicon dioxide.

In these examples, the wiping method is used to simulate the cycle tests to obtain the moisture-absorbing effects of the moisture-absorbing electrode (moisture-absorbing conductive film) containing different proportions of moisture-absorbing materials (1 wt %, 3 wt % and 5 wt %). The examples include the following steps. Initially, the film is placed in a natural environment for a period of time, so that the moisture-absorbing materials (lithium chloride, attapulgite or silicon dioxide) can absorb water molecules in the air. Then, a wipe is applied to wipe the moisture-absorbing materials on the surface of the film. These steps can simulate the effect of repeatedly using the film. Afterwards, the film is placed in an oven for drying so as to restore the moisture-absorbing property of the moisture-absorbing materials. Then, these steps are repeated to perform the moisture absorption cycle test.

As shown in FIGS. 4A to 4C, it is found that the moisture-absorbing electrodes (moisture-absorbing conductive films) containing lithium chloride, attapulgite or silicon dioxide with different proportions can be repeatedly used for multiple times, and they all have good durability.

Referring to the following Table 1, which shows the changes in weights and resistances of moisture-absorbing materials (with different proportions) before and after moisture absorption. In these cases, three moisture-absorbing materials (lithium chloride, attapulgite, and silicon dioxide) all have good moisture-absorbing abilities. In addition, after absorbing moisture for a period of time, the resistance values of various moisture-absorbing materials do not change much. When the moisture-absorbing material is silicon dioxide, the resistance values of the moisture-absorbing materials (with silicon dioxide of some proportions) become smaller after moisture absorption, which is beneficial to signal transmission. In addition, it can also be seen from Table 1 that the moisture-absorbing effect can be achieved after adding the moisture-absorbing materials. Moreover, with the same content of the moisture-absorbing materials, the moisture-absorbing effect of the case of adding silicon dioxide is more obvious than that of adding attapulgite, but the resistance value of the case of adding attapulgite is more stable than the others.

TABLE 1

| | | | | weight | resistance |
|---|---|---|---|---|---|
| | | | | change | change |
| | | resistance | resistance | before and | before and |
| | | before | after | after | after |
| | | moisture | moisture | moisture | moisture |
| | | absorption | absorption | absorption | absorption |
| item | wt % | (Ω/cm) | (Ω/cm) | (%) | (%) |
| carbon | 100 | 72.5 | 98.2 | NA | +35.45 |
| paste | | | | | |
| lithium | 1 | 72.8 | 77.8 | 110.72 | +6.87 |
| chloride | 3 | 105.2 | 115.4 | 114.72 | +9.70 |
| | 5 | 108.4 | 121.6 | 117.81 | +12.18 |
| | 10 | 153.4 | 185.1 | 116.87 | +20.66 |
| silicon | 0.1 | 144.3 | 149.6 | 123.51 | +5.3 |
| dioxide | 0.3 | 99.5 | 105.9 | 120.74 | +6.4 |
| | 0.5 | 133.2 | 177.3 | 117.30 | +44.1 |
| | 1 | 81.8 | 79.1 | 121.79 | −3.30 |
| | 3 | 108.4 | 81.5 | 117.21 | −24.82 |
| | 5 | 124.5 | 82.4 | 112.79 | −33.87 |
| | 10 | 126.4 | 83.8 | 111.69 | −33.70 |
| attapulgite | 0.1 | 69.34 | 76.53 | 116.48 | +7.19 |
| | 0.3 | 89.53 | 94.8 | 115.90 | +5.3 |
| | 0.5 | 68.94 | 77.98 | 115.17 | +9.04 |
| | 1 | 106.4 | 128.2 | 117.38 | +20.49 |
| | 3 | 133 | 144.4 | 116.34 | +8.57 |
| | 5 | 155.2 | 162.3 | 112.80 | +4.57 |

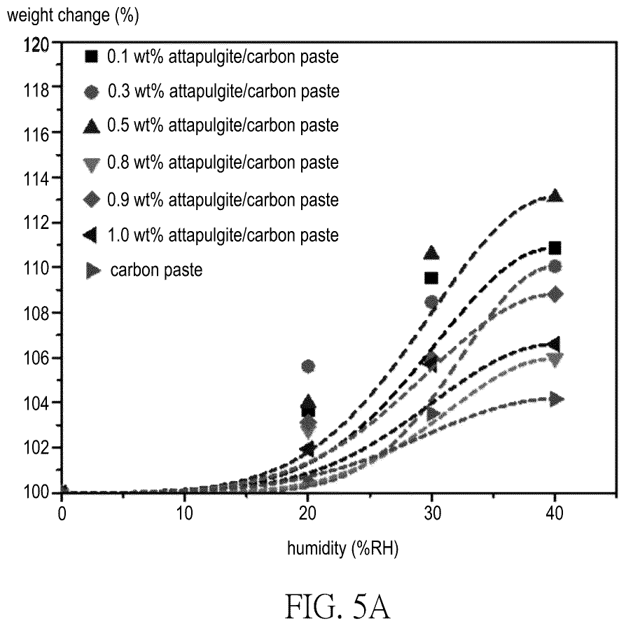

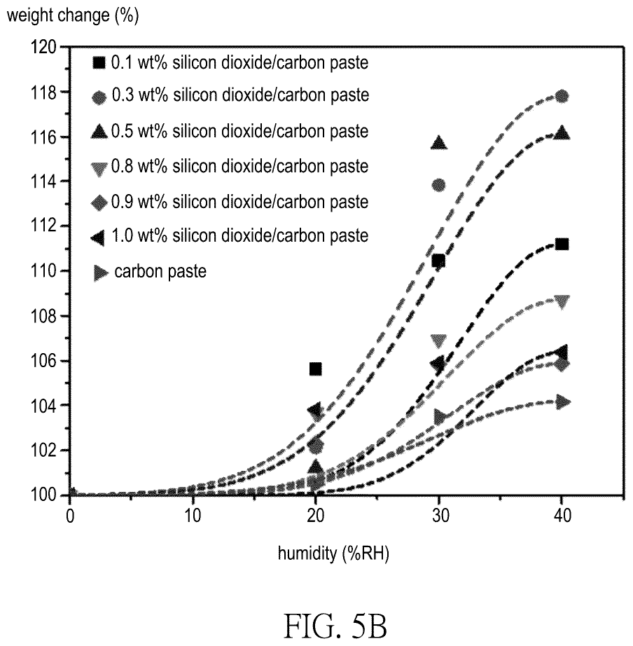

FIGS. 5A and 5B are schematic diagrams showing the results of the moisture-absorbing effects of carbon paste and different moisture-absorbing materials (with different proportions) in different humidity environments. Referring to FIGS. 5A and 5B, the carbon paste and the moisture-absorbing material (attapulgite or silicon dioxide) are placed in the oven under different humidity (20%, 30% or 40%) for 48 hours. This step can simulate the moisture-absorbing saturation of the materials and predict the moisture-absorbing state of the materials in different humidity environments.

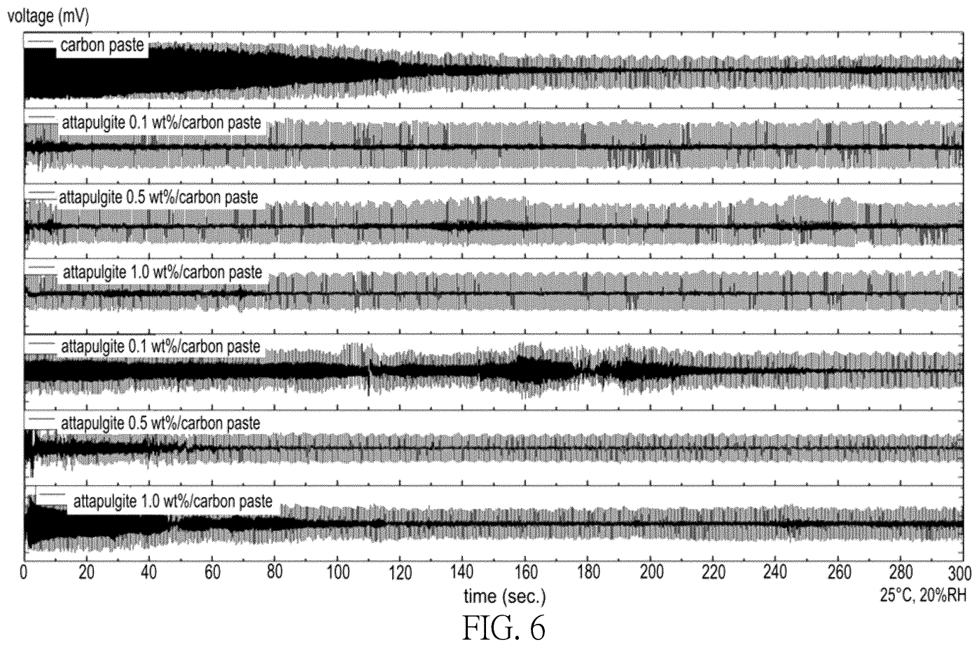

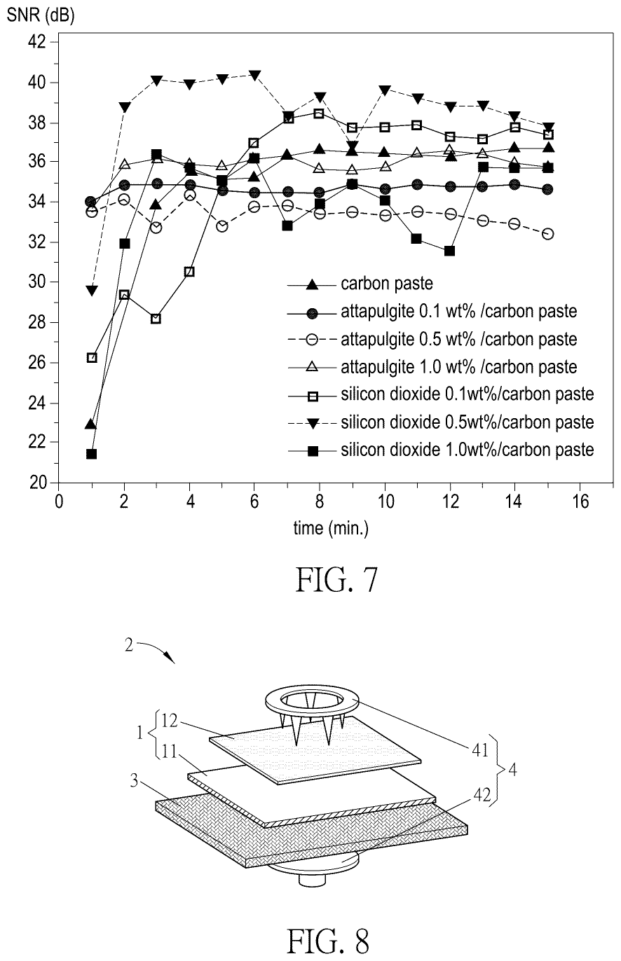

FIG. 6 is a schematic diagram showing the electrocardiogram (ECG) signals measured by the electrode patches made of different materials and proportions, and FIG. 7 is a schematic diagram of the signal-to-noise ratio (SNR) plotted from the measurement results of FIG. 6. In the measurement results of FIG. 6, the waveforms were measured in an environment with constant temperature and humidity (25° C., 20% RH). In addition, in FIG. 7, a high SNR (e.g. greater than 30 dB) indicates that the measured signal strength is much stronger than the noise, which means that the ECG signal is very clear and easy to be detected and interpreted. A high SNR helps improve the signal accuracy and reliability. In these examples, the electrode patch made of pure carbon paste can be realized as the conventional dry electrode.

It can be seen from FIGS. 6 and 7 that the ECG signal measured by the dry electrode patch containing only carbon paste has a lot of noise (low SNR) in the front three minutes, and the ECG signal gradually becomes stable three minutes later. In another case, the ECG signal measured by the moisture-absorbing dry electrode patch containing attapulgite can quickly reach stable and has high SNR, which can improve the accuracy and reliability of the signal and can fit the desired effect. In yet another case, compared to the dry electrode patch containing only carbon paste, the ECG signal measured by the moisture-absorbing dry electrode patch containing silicon dioxide can reach stable earlier, and the SNR thereof is also higher than that of the dry electrode patch containing only carbon paste. Thus, the moisture-absorbing effect of the moisture-absorbing dry electrode patch containing silicon dioxide is better than that of the dry electrode patch containing only carbon paste. In practice, the moisture-absorbing dry electrode with silicon dioxide of 0.5 wt % has a higher SNR and can meet the requirement much more.

In the moisture-absorbing dry electrode of the above embodiment, the moisture-absorbing material is added to the conductive film to absorb moisture. This configuration can increase the contact area between the electrode and human skin, thereby improving signal reception and allowing repeated use. Therefore, compared with the conventional electrode patches, the moisture-absorbing dry electrode of this embodiment combines the convenience and stability as the conventional dry electrodes and the good signal reception and skin comfortable as the conventional wet electrodes, so that it can be conveniently used and does not require additional electrolysis or gel-like substance. Thus, the long-term wearing of the moisture-absorbing dry electrode of this embodiment will not cause skin irritation or allergic reactions. In addition, the moisture-absorbing dry electrode of this embodiment has good stability, so that it can achieve better resistance to movement or other interferences, and can maintain signal quality more stably. Moreover, in high-activity scenes such as sports and fitness, the moisture-absorbing material can absorb sweat and other secretions to maintain good contact between the electrode patch and the skin, thereby ensuring signal quality and stability and meeting the needs of users.

FIG. 8 is a schematic diagram showing a part of a smart clothing according to an embodiment of the present disclosure. In this embodiment, FIG. 8 shows the moisture-absorbing dry electrode 1, which connects to a clothing body 3.

As shown in FIG. 8, a smart clothing 2 of this embodiment can be used to measure physiological signals of human body. The smart clothing 2 include a clothing body 3 and a moisture-absorbing dry electrode 1. In this embodiment, the clothing body 3 can be worn on the human body, and the material of the clothing body 3 is not limited. The moisture-absorbing dry electrode 1 can be arranged on the inner side of the clothing body 3 and contact the human body to measure physiological signals. In this embodiment, the moisture-absorbing dry electrode 1 is fixed on the inner side of the clothing body 3 by, for example, a five-prong metal snap button set 4 and is attached to the human body. The five-prong metal snap button set 4 includes a prong part 41 and a stub part 42. The prong part 41 and the stub part 42 are correspondingly fastened to fix the moisture-absorbing dry electrode 1 on the clothing body 3. The moisture-absorbing dry electrode 1 and the clothing body 3 are disposed between the prong part 41 and the stub part 42. In this case, the advantage of using the five-prong metal snap button set 4 to fix the moisture-absorbing dry electrode 1 to the clothing body 3 is that the five-prong metal snap button set 4 can be used as an adapter for connecting measuring the instrument of physiological signals (e.g. EEG, ECG or EMG), or the five-prong metal snap button set 4 can be used as the electrical connection terminal of the smart clothing 2. In different embodiments, the moisture-absorbing dry electrode 1 can also be fixed on the inner side of the clothing body 3 by using, for example, locking, adhering, buckling, or any of other connection methods, and this disclosure is not limited thereto.

The moisture-absorbing dry electrode 1 includes a substrate 11 and a moisture-absorbing electrode 12. The substrate 11 is connected to the clothing body 3 and has a surface 111 away from the clothing body 3. The moisture-absorbing electrode 12 includes at least one moisture-absorbing conductive film 121, which is used for adsorbing moisture and is disposed on the surface 111 of the substrate 11. The material of the moisture-absorbing conductive film 121 may include uniformly mixed carbon paste, a moisture-absorbing material and a cross-linking agent, and the moisture-absorbing material may include a nanomaterial or an inorganic adsorbent material, or a combination thereof. In this case, the other technical features of the moisture-absorbing dry electrode 1 have been described in detail above, and the detailed description thereof will be omitted here. It can be understood that in different embodiments, the moisture-absorbing dry electrode 1a can be used to replace the above-mentioned moisture-absorbing dry electrode 1, and it can also achieve the good moisture-absorbing effect.

In summary, in the moisture-absorbing dry electrode and smart clothing of the present disclosure invention, the conductive film is added with the moisture-absorbing material for absorbing moisture, so that the contact area between the electrode and human skin can be increased, thereby improving signal reception. Compared with the conventional electrode patches, the moisture-absorbing dry electrode and smart clothing of this disclosure have the convenience and stability as the conventional dry electrodes and the good signal reception and skin comfortable as the conventional wet electrodes, so that they can be conveniently used and do not require additional electrolysis or gel-like substance. Therefore, the long-term wearing of the smart clothing will not cause skin irritation or allergic reactions. In addition, the moisture-absorbing dry electrode of the present disclosure has good stability, so that it can achieve better resistance to movement or other interferences, and can maintain signal quality more stably. Moreover, in high-activity scenes such as sports and fitness, the moisture-absorbing material can absorb sweat and other secretions to maintain good contact between the electrode patch and the skin, thereby ensuring signal quality and stability and meeting the needs of users.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A moisture-absorbing dry electrode, comprising:
a substrate having a surface; and
a moisture-absorbing electrode, comprising at least one moisture-absorbing conductive film configured to absorb moisture, wherein the moisture-absorbing conductive film is disposed on the surface of the substrate;
wherein, a material of the moisture-absorbing conductive film comprises a uniform mixture of carbon paste, a moisture-absorbing material and a cross-linking agent, and the moisture-absorbing material comprises a nanomaterial or an inorganic adsorbent material, or a combination thereof.

2. The moisture-absorbing dry electrode of claim 1, wherein a material of the substrate comprises a polymer material.

3. The moisture-absorbing dry electrode of claim 1, wherein the carbon paste is a mixture of a solvent and one or any combinations of graphene, carbon nanotubes and carbon black.

4. The moisture-absorbing dry electrode of claim 1, wherein a content of the moisture-absorbing material is between 0.1 and 50 weight percentage of the carbon paste.

5. The moisture-absorbing dry electrode of claim 1, wherein the nanomaterial comprises attapulgite, silicon dioxide, zeolite, porous carbon, activated carbon, covalent organic framework material, metal organic framework material or porous organic polymer, or any combinations thereof.

6. The moisture-absorbing dry electrode of claim 1, wherein the inorganic adsorbent material comprises metal salt or activated alumina, or a combination thereof.

7. The moisture-absorbing dry electrode of claim 1, wherein a thickness of the moisture-absorbing conductive film is between 1 μm and 2000 μm.

8. The moisture-absorbing dry electrode of claim 1, wherein the moisture-absorbing electrode comprises multiple of the moisture-absorbing conductive films, the moisture-absorbing conductive films are stacked and arranged on the surface of the substrate, and a total thickness of the moisture-absorbing conductive films is between 1 μm and 2000 μm.

9. The moisture-absorbing dry electrode of claim 1, wherein the moisture-absorbing material comprises lithium chloride, and a moisture-absorbing ability of the moisture-absorbing electrode has a positive correlation with a content of the lithium chloride in the moisture-absorbing material.

10. A smart clothing, applied to measure a physiological signal, comprising:
a clothing body configured to be worn on a human body; and
a moisture-absorbing dry electrode arranged at an inner side of the clothing body and configured to contact the human body for measuring the physiological signal, wherein the moisture-absorbing dry electrode comprises a substrate and a moisture-absorbing electrode, the substrate is attached to the clothing body and has a surface away from the clothing body, and the moisture-absorbing electrode comprises at least one moisture-absorbing conductive film configured to absorb moisture and is disposed on the surface of the substrate;
wherein, a material of the moisture-absorbing conductive film comprises a uniform mixture of carbon paste, a moisture-absorbing material and a cross-linking agent, and the moisture-absorbing material comprises a nanomaterial or an inorganic adsorbent material, or a combination thereof.

11. The smart clothing of claim 10, wherein a material of the substrate comprises a polymer material.

12. The smart clothing of claim 10, wherein the carbon paste is a mixture of a solvent and one or any combinations of graphene, carbon nanotubes and carbon black.

13. The smart clothing of claim 10, wherein a content of the moisture-absorbing material is between 0.1 and 50 weight percentage of the carbon paste.

14. The smart clothing of claim 10, wherein the nanomaterial comprises attapulgite, silicon dioxide, zeolite, porous carbon, activated carbon, covalent organic framework material, metal organic framework material or porous organic polymer, or any combinations thereof.

15. The smart clothing of claim 10, wherein the inorganic adsorbent material comprises metal salt or activated alumina, or a combination thereof.

16. The smart clothing of claim 10, wherein a thickness of the moisture-absorbing conductive film is between 1 μm and 2000 μm.

17. The smart clothing of claim 10, wherein the moisture-absorbing electrode comprises multiple of the moisture-absorbing conductive films, the moisture-absorbing conductive films are stacked and arranged on the surface of the substrate, and a total thickness of the moisture-absorbing conductive films is between 1 μm and 2000 μm.

18. The smart clothing of claim 10, wherein the moisture-absorbing material comprises lithium chloride, and a moisture-absorbing ability of the moisture-absorbing electrode has a positive correlation with a content of the lithium chloride in the moisture-absorbing material.
